(19) United States
(12) Patent Application Publication
(10) Pub. No.: US 2006/0141024 A1
(43) Pub. Date: Jun. 29, 2006

(54) Synergistic combination kit of alpha,beta-arteether, sulfadoxin and pyrimethamine for treatment of severe/multi-drug resistant cerebral malaria

(75) Inventors: Renu Tripathi, Uttar Pradesh (IN); Sunil K. Puri, Uttar Pradesh (IN); Jagdishwar S. Srivastava, Uttar Pradesh (IN); Satyawan Singh, Uttar Pradesh (IN); Omkar P. Asthana, Uttar Pradesh (IN); Anil K. Dwivedi, Uttar Pradesh (IN)

Correspondence Address: NIXON & VANDERHYE, PC, 901 NORTH GLEBE ROAD, 11TH FLOOR, ARLINGTON, VA, 22203, US

(73) Assignee: COUNCIL OF SIENTIFIC AND INDUSTRIAL RESEARCH, New Delhi (IN)

(21) Appl. No.: 11/022,929

(22) Filed: Dec. 28, 2004

(51) Int. Cl. A61K 9/48; A61K 9/20; A61K 31/505; A61K 31/335

(52) U.S. Cl. 424/451; 424/464; 514/275; 514/452

(57) ABSTRACT

The present invention relates to a method for preparation of synergistic combination kits of α,β-arteether, sulfadoxin and pyrimethamine for the treatment of severe/multi-drug resistant cerebral malaria.

### FIELD OF INVENTION

The present invention relates to a method for preparation of synergistic combination kits of α,β-arteether, sulfadoxin and pyrimethanine for the treatment of severe/multi-drug resistant cerebral malaria. These combination kits are for the use in the treatment of malaria. Particularly, the invention relates to combination kits comprising anti-malarial agents, α,β-arteether, sulfadoxin and pyrimethamine. More particularly the present invention relates to the use of the combination kits containing anti-malarial agents α,β-arteether, sulfadoxin and pyrimethamine against cerebral malaria caused by multi-drug resistant Plasmodium falciparum for better patient compliance. The present invention further relates to a method for the treatment of malaria caused by multi-drug resistant Plasmodium falciparum.

### BACKGROUND OF INVENTION

Malaria, caused by a parasitic protozoan called Plasmodium, is one of the most serious and complex tropical parasitic diseases. Generally human malaria is caused by four species of malaria parasites—Plasmodium falciparum, Plasmodium vivax, Plasmodium ovale and Plasmodium malariae. Out of these Plasmodium flaciparum and Plasmodium vivax are wide spread and cause significant mortality and morbidity associated with these types of infections.

A number of drugs ranging from those of natural origin to synthetic ones have been developed for the treatment of malaria. Quinine and artemisinin are the commonly known drugs of natural origin, which are mostly used for the treatment of malaria. A number of synthetic anti-malarial drugs such as chloroquine, mefloquine, primaquine, halofantrin, amodiaquine, proguanil, atovaquine, maloprim are known in the literature.

In recent years drug resistant malaria has become one of the most serious problems in malaria control. Drug resistance necessitates the use of drugs, which are more expensive and may have dangerous side effects.

Cerebral malaria remains a major cause of mortality in many tropical areas of the world. According to WHO P. falciparum malaria is becoming increasing resistant to chloroquine, sulfadoxine-pyrimethamine and mefloquine (Eyles et al. 1963, Am. J. Trop. Med. Hyg. 12, 935-840, WHO Tech Report Series No. 711, 1984; Boudreau et. al. 1982 Lancet II, 1335, Nosten et al. 1991, Lancet 337, 1140-1143; Hurwitz et al. 1981, Lancet 1, 1068-1070, Timmermanns et. al. 1982, Lancet 1, 1181).

During the 1960s several strains of the malarial parasite Plasmodium falciparum developed resistance to the synthetic drugs particularly the highly valued chloroquine. The parasite mostly remained sensitive, however, to quinine, which had to be reinstated inspite of the side effects at larger doses. Although quinine resistance is also emerging along Thailand-Myanamar border, still the quinine and tetracycline combinations remains over 80% effective in practice (Vanijanonta et al. Lancet 1992, 339-369). Besides, seven day regimen of quinine and tetracycline was found to be effective in treating P. falciparum cases which were resistant to artesunate, artemether and mefloquine combined therapy. A number of antimalarial combinations are already known in the malarial chemotherapy. The use of drug combinations in antimalarial treatment not only delays the onset of drug resistance, but also accelerates recovery and increases cure rates. For example a combination of amodiaquine and tetracycline, a combination of sulfadoxine and pyrimethamine known as fansidar, are known therapies for the treatment of P. falciparum. In 1976, the World Health Organization recommended Fansidar as the best chemo prophylactic and chemotherapeutic agents against human malaria.

Mefloquine was reported to produce neuropsychiatric side effects in adults who developed tonic clonic fits. Besides psychosis, delusions and hallucinations, anxiety sleep disturbances were also reported after mefloquine treatment (Drugs 1990, 39,160-169).

Halofantrine is more effective but rather high doses of the drug are now required to control resistant P. falciparum (Brasseur et al. Lancet 341, 1993, 901-2) which could lead to increase risk of cardiotoxicity of this new antimalarial including sinus bradycardia, sinus arrhythmia tall peak, T. waves, QT interval prolongation, ectopic beats (Karbwang et al. Lancet 342, 1993, 501, Wilding et al. Lancet.342, 1993, 55, Kremsner et al. Am. J. Trop. Med. Hyg. 50 (6), 1994, 790-795) which has imposed great limitation on the antimalarial potential of this drug.

Multi-drug-resistance in P. falciparum malaria and high level of P. vivax resistant strains are posing a major threat to countries in South East Asia, Africa and Southern America. Most seriously affected areas include Thia-Myanamar Burmese border, where resistance to nearly all available drugs (chloroquine, sulfadoxine-pyrimethamine, mefloquine, quinine) has already been established, (WHO Report on Infectious Diseases, 1999, WHO/CDS/99. 1).

The cheapest and most readily available antimalarials are increasingly ineffective and, in its Roll Back Malaria program, the WHO is urging countries to switch to new combination therapies when there is strong evidence that exstiag conventional medicines are no longer working.

Combination of atovaquone and proguanil (U.S. Pat. No. 5,998,449) describes a method for the treatment of malaria. Combination of fenozan with another anti-malarial agents selected from artemisinin, sodium artesunate, chloroquine, mefloquine (U.S. Pat. No. 5,834,505) is described for the prophylactic and curative treatment of malaria.

With the emergence of strains P. falciparum resistant to chloroquine and quinine, alternative antimalarial is required. The artemisinin, obtained from the plant Artemisia anua, and its derivatives are rapidly effective in severe malaria. Artemisinin drugs are very effective in causing rapid parasite clearance and fever resolution in malaria patient.

Artemisinin compounds have been evaluated in several centers and are found to be effective, and safe (Hien T. T., White N. J. Lancet 341, 1993, 603-08, Tripathi, et. al. Am. J. Trop Med Hyg. 43 (5), 1990, 571-75, Miskra S. K., Asthana O. P. et al., Trans R Soc Trop Med Hyg 89, 1995, 299-301.) Many authors have substantiated its rapid schizonticidal action. Usefulness of the drug in severe malaria has also been demonstrated.

α,β-Arteether is an ethyl ether derivative of dihydroartermisinine, which is dihydro derivative of artemisinine. Artemisinine is extracted from Artemisia annua, a plant long known in traditional Chinese medicine for its anti malarial properties. α,β Arteether (30:70) (intramuscular) is one of the artemisinin derivatives that has been developed in India and shows antimalarial activity against chloroquinine, mefloquine and quinine resistant P. yoelii nigeriensis and cures experimental cerebral malaria infections (P. knowlesi/P. fragile in rhesus monkey model, Dutta et al., Pharmacol Res. 21, 1989, 415-19; Dutta and Tripathi, Japan. J. Trop. Med. Hyg. 24, 1996, 65-69; Tripathi et al. Exp. Parasitol. 87, 1997, 290-292; Bajpai et al Trans. R. Soc. Trop. Med. Hyg. 83, 1989, 484).

α,β-Arteether is relatively safe and its LD₅₀ dose is 1250 mg/kg, im in swiss mice as compared to artemether (LD₅₀ 263 mg/kg,im) and artesunate (LD₅₀ 475 mg/kg,imn) (J. Trad. Chinese Med 2 (1), 1982, 31-38).

Two hundred and sixty seven patients of uncomplicated P. falciparum malaria completed study in a multi-centric phase III clinical trial of arteether (150 mg i.m. daily for three days). (Asthana et. el. J. A P. I. 49, 2001, 692-696). The cure rate was 97% with fever clearance time between 1-7 days and parasite clearance time between 1-3 days. Clinical Efficacy of α,β-arteether in patients of falciparum malaria was reported by Mishra S. K., Asthama O. P. et. al. (Trans. R. Soc. Trop. Med. Hyg. 89, 1995, 299-301). A total of 211 patients of complicated P. falciparum malaria showed that fever clearance time range between 24-168 hours. The overall cure rate was 93%. (Asthana et. el. J.A.P.I. 49, 2001, 1155-1160). The report of rapid parasite clearance, coma resolution and decreased mortality in cerebral malaria in children has led to increase interest of α,β-arteether as the first line drugs.

The WHO (1984) had accorded high priority to the development of fast acting artemisinin derivatives as blood schizontocides for the emergency treatment of cerebral malaria as well as for the control of multiple drug resistant cases of Plasmodium falciparum.

Rapid coma resolution with artemether in cerebral malaria was reported in Lancet 341, 1993, 61-2. Open randomized comparative study of artemether and intravenous quinine in cerebral malaria in children was reported in Trans. R. Soc. Trop. Med. Hyg. (87, 1993, 564-6). A trial of artemether or quinine in children with cerebral malaria were also carried out. (Van Hensbroek M. B., Onyiorah E., Jaffer S. et al. New Eng. J Med. 335, 1996, 69-75, New Eng J Med 335, 1996 124-26, and New Eng J Med 335, 1996, 19234, Salako L A, Walker O. et al. Trans R Soc Trop Med Hyg 88, suppl I:S 1994, 13-15, Sowunmi A, Oduola A M, Acta Trop 61(1), 1996, 57-63). Randomized comparison of intra-mscular artemether and intravenous quinine in adult Melanesian patients with severe or complicated P. falciparum in Papua New Guinea were carried out. (Seaton R A, Trevett A J, Wemberi J P, et al. Ann Trop Med. Parasitol. 92, 1998, 133-9). The Myanamar study in 141 cerebral malaria cases with intravenous artemisinin plus oral mefloquine decreased mortality and rapid parasite clearance in comparison to intravenous quinine with oral tetracycline. (Win K Than M, Thwe Y Bull WHO 70, 1992, 777-822). The Vietnam study in 79 comatose cerebral malaria cases demonstrated that there was rapid reduction in parasiteamia [16 hrs for artemisiain versus 34.5 hrs for intravenous quinine]. But they did not notice significant reduction in the duration of coma or mortality. (Hien T T, Arnol K, Vien N N, et al. Trans R Soc Trop Med Hyg 86, 1992, 582-3.). Martin Danis et al demonstrated a Meta analysis using individual patient data of trials comprising artemether with quinine in the treatment of severe falciparum malaria (Trans R Soc Trop Med Hyg; 95, 2001, 637-650)

The World Health Organization is urging developing countries to tackle rising levels of resistance to antimalarial medicines, particularly with the use of Artemisinin-based Combination Therapies (ACTs) ACTs combine two medicines that work in different ways. According to the WHO, it is unlikely that the malaria parasite would evolve to resist these combinations and they also kill the malaria parasite very fast with few side effects.

The WHO points out that the artemether-lumefantrine combination is the only product so far which combines an artemisinin and non-artemisinin compound in a single tablet. But it also recommends other combinations of artemisinin compounds with currently used medicines, such as amodiaquine, for use where these medicines are still effective.

Artemisinin derivatives are the most wanted partner drugs for combination chemotherapy for malaria. Among those artesunate is widely used derivative. Newton et al (2001) had reported that artesunate 12 mg/kg (iv) versus artesunate and sequential treatment with quinine 30 mg/kg (iv) to falciparum malaria patients. There was no significant difference between the two groups. Artemsinin and quinine for uncomplicated malaria was also studied (Peter et al, 2000). Artemisinine 20 mg/kg×3/5 ays showed 38% and 15% recrudescence respectively whereas with quinine 10 mg/kg×7 days showed 16% recrudescence. Again it shows that 5 days artemnisinin are equally effective as oral quinine. In vitro studies with some strains also show synergism between quinine and artenisinin but synergism was not consistent.

Recent clinical trials conducted in Indonesia show that artesunate (4 mg/kg×3 day, oral) in combination with SP (Sulfadoxin+pyrimethamine) produced 100% curative effect on day 14 and the same was 95.6% on day 28 and they were compared with 87.0% and 84.8% success rate on day 14 & 28 respectively with SP (Sulfadoxin+pyrimethamine) alone (Am. J. Trop. Med. & Hyg 65(4), 2001, 309-317). Another report (Lancet, 363, 2004, 9-17) again confirmed the addition of 3 days of artesunate to standard treatment with Fansidar substantially reduce treatment failure, recrudescence and gametocyte carriage. In western Kenya, this combination was trialed in children (<5 years) and treatment failures rates by day 14 were 25.5% in SP alone, 9.4% in 3-day artesunate treatment group. Corresponding rates by day 28 were 42.5% in SP alone and 20.7% in combination group. (Charles et al 2003) Trans. Roy. Trop. Med. & Hyg.97, 585-591).

Chorproguanil and dapsone is also a new and very cheap antimalarial combination, its single and 3 day regimens are available. It is a good substitute of SP because of its rapid elimination and to reduce the rate of emergence of antifolate resistance in Africa and for affordable salvage therapy in case of SP failure (Amukoye et al 1997). But the present regimen of chlorproguanil-dapsone remains effective for no longer than 6 days.

Any combination, which reduces the dose and duration of treatment or which reduces the recurrence, will automatically reduce the side effects. Therefore, there is a long felt need for a more practical, effective, patient compliant and safe remedy for the radical cure of P. falciparum malarial infection.

Objects of the Invention

The main object of the invention is to provide a synergistic combination kit for the treatment of severe/multi-drug resistant cerebral malaria which is practical, effective, safe and patient compliant.

### SUMMARY OF THE INVENTION

The inventors have found that the above object can be fulfilled by providing a treatment regime consisting of regulated use of sulfadoxin, pyrimethamine and α,β-arteether over a period of three to five days. Although artesunate with SP are being used in clinical trials but artesunate have poor stability and less LD₅₀ as compared to arteether (α/β). Therefore the combination kit of the present invention (α/βarteether+SP) produces better clinical effect and is more stable and less toxic than artesunate+SP.

Accordingly, the present invention provides a combination kit for the treatment of malaria caused by P. falciparum for a period of two to five days schedule, the kit comprising:
a. individual dose of anti-malarial agents sulfadoidne and pyrimetbiine, and
b. individual dose of anti-malarial agent α,β arteether;
c. instruction manual for the administration of the three antimalarial drugs.

In one embodiment of the invention, the sulfadoxine and pyrimethamine are used in a form selected from the group consisting of capsules, suppositories and tablets.

In another embodiment of the invention, the α,β arteether is taken in ground nut oil filled in a injection vial or soft gelatin capsule or in the form of capsules or tablets.

In another embodiment of the invention, the kit comprises:
a. three capsules/suppositories/tablets containing 500 mg of sulfadoxine and 25 mg of pyrimetharmine; and
b. three injection each containing 150 mg of α/β arteether or fifteen capsules/tablets containing 150-250 mg of α/β arteether.

In another embodiment of the invention, the kit is in the form of one or more strips in which the anti-malarial agents are packed.

In another embodiment of the invention, the kit is in the form of a small carton wherein the instruction is in form of printed instruction provided inside the carton.

In another embodiment of the invention, the instructions are in the form of prints on the carton and/or on the strip or strips.

The present invention also provides a method for the treatment of malaria caused by P. falciparum comprising administering to a subject malarial medication contained in a combination kit which in turn comprises:
a. individual dose of anti-malarial agents sulfadoxine and pyrimethamine, and
b. individual dose of anti-malarial agent α,β arteether;
c. instruction manual for the admuinistration of the three antimalarial drugs.

In one embodiment of the invention, the sulfadoxine and pyrimethamine are used in a form selected from the group consisting of capsules, suppositories and tablets.

In another embodiment of the invention, the α,β arteether is taken in ground nut oil filled in a injection vial or soft gelatin capsule or in the form of capsules or tablets.

In another embodiment of the invention, the kit comprises:
a. three capsules/suppositories/tablets containing 500 mg of sulfadoxine and 25 mg of pyrimethatamine; and
b. three injection each containing 150 mg of α/β arteether or fifteen capsules/tablets containing 150-250 mg of α/β arteether.

In a further embodiment of the invention, the method of administration comprises
i) administering three capsules/suppositories/tablets containing 500 mg of sulfadoxine and and 25 mg of pyrimethanrine on day one;
ii) administering one i.m. injection at a time, each containing 150 mg of α,β arteether per day for two to three days or alternatively administer three tablets/capsules each containing 150 mg-250 mg of α,β arteether per day for three-five days by oral route.

In another embodiment of the invention, the method comprises administering a first anti-malarial agent sulfadoxin, second antimalarial Pyrimethamine for single day and third anti-malarial agent α/β arteether in predetermined doses and predetermined dose schedule for two to three/five day therapy.

### DETAILED DESCRIPTION OF THE INVENTION

The curative dose of α,β arteether against P. yoelii nigeriensis in gwiss mice (a murine model equivalent to P. falciparum in human) is 7.5 mg/kg for three days. Sulfadoxin/pyrimethtnine combination is usually given for P. falciparum infection, to adult humans as a single dose of 1500 mg sulfadoxin and 75 mg pyremethamine (usually in 3 tablets of 500 mg sulfadoxin and 25 mg pyremethamine) (practical chemotherapy of malaria, WHO, Geneva 1990). For children if they are below 40 kg, only 2 tablets are recommended. In vitro studies show 100% inhibition with <5.0 mmol/lit and 62.5 μmol/lit of sulfadoxin and pyrimethamine respectively. Sulfadoxin remains higher in plasma than in whole blood because high protein binding capacity of the drug which is acid and does not accumulate in erythrocyte. Although resistance to Fansidar is documented in expatriates in East Africa (Schapira et al. 1986), the drug is reported to be generally effective in the indigenous population (Spencer et al. 1984) and it is treated as second line of drug and is the only affordable drug for chloroquine resistant cases in developing countries. In combination the proposed doses of α,β arteether plus sulfadoxin and pyrimethamine for humans would be 150 mg×3 (im injections) or 450-750 mg×5 (oral dose) of α,β arteether with three tablets of 500 mg sulfadoxin and 25 mg of pyrimethamine for P. falciparum. The proposed combination therapy will certainly reduce the incidence of recrudescence and new infection in high transmission areas.

On the basis of these studies the curative treatment schedule for the treatment of multi drug resistant P. falciparum will be 1500 mg sulfadoxin and 75 mg of pyrimethamine on day one and 150 mg (im injection) of α,β arteether once in a day for total three days or 1500 mg sulfadoxin and 75 mg of pyrimethamine on day one and 450-750 mg (oral dose) of α,β arteether one in a day for total five days.

It has also been found that the treatment regime may be executed most effectively and in a user friendly manner by providing a combination kit which comprises three antimalarial agents, namely sulfadoxin, pyrimethamine and α,β-arteether and an instruction material containing instructions for the administration of three antimalarial agents during the period of treatment.

Mechanisms of Drugs Action

Fansidar is a combination of two antifolate drugs i.e. sulfadoxin and pyrimethamine (20:1 ratio i.e.500 mg+25 mg). This drug combination is often used and is second line drug for the treatment of chloroquine resistant plasmodium falciparun malaria (Hall et al. 1977, Dobenstyn et al 1979). Sulfadoxin is a long acting sulfonamide; it inhibits dihydropteroate synthase whereas pyrimethamine inhibits dihydrofolate dehydrogenase enzymes. These enzymes control successive steps in the parasite's folic acid cycle and, when used together, potentiate each other. Also fansimef, a combination of mefloquiae with sulfadoxine and pyrimethanine is used against multi-drug resistant strains of P. falciparum

Most of the artemisinin compounds including α,β arteether metabolized into dihydroartemisinin (DHA) in the body and, which is responsible for antimalarial activity. These compounds contain stable endoperoxide bridge. The antimalarial activity of artemisinin is dependent on the cleavage of the endoperoxide by intraparasitic heme. The cleaved endoperoxide ultimately becomes a carbon centered free radical, which then functions as an alkylating agent, reacting with both heme and parasitic proteins (but not DNA).

In P. falciparum, one of the principal alkylation target is the translationally controlled tumor protein (DHA-TCTP) homolog. Some intraparasitic TCTP is situated in the membrane surrounding the heme-rich food vacuole, where heme could catalyse the formation of drug-protein (DHA-TCTP) adduct and inhibit the parasite's growth.

Thus combining the three drug will increase the sites of action on the parasites and thus will be more effective in controlling them. The present invention therefore relates to a combination kit for the treatment of P. falciparum malaria for a period of three to five days. The kit comprises:
1. A predetermined dose of anti-malarial agents namely sulfadoxin and pyrimethamine;
2. A predetermined dose of a third anti-malarial agent namely α,β arteether.
3. An instruction manual containing instructions for administering the three antimalarial agents during the treatment period.

The present invention also relates to a method of treatment of malaria caused by P. falciparum comprising administering antimalarial agents sulfadoxin and pyrimethamine and followed by anti-malarial agent, α,β-arteether in a predetermined doses and in a predetermined sequence for a period of three to five days.

As explained above, the combination kit for the treatment of malaria caused by P. falciparum for a period of three to five days schedule essentially comprises:
1. Individual dose of anti-malarial agents, sulfadoxin and pyrimethamine in the form of tablet/capsules and;
2. Individual dose of the anti-malarial agent, α,β arteether in groundnut oil in the form of injection or capsule/tablet.
3. Instruction manual for the administration of the three antimalarial drugs.

For example, the kit can comprise:
1. Three capsules/tablets each containing 500 mg of sulfadoxin and 25 mg of pyrimethamme.
2. Three injections of α,β arteether, each containing 150 mg of drug or fifteen capsule/tablet of α/β arteether, each containing 150 to 250 mg of drug.
3. Instruction material having directions to administer treatment in the following manner.
   a. To administer three capsules/tablets each containing 500 mg of sulfadoxin and 25 mg of pyrimetharnine on day one.
   b. To administer one i.m. injection at a time, each containing 150 mg of α,β arteether per day for three days or alternatively administer three capsule/tablet each containing 150 to 250 mg of α,β arteether per day for five days.

The method of treatment of malaria caused by P. falciparum for a period of three to five days comprises:
a. administering three capsules/tablets each containing 500 mg of sulfadoxin and 25 mg of pyrimethamine.
b. administering one i.m. injection at a time, each containing 150 mg of α,β-arteether per day for three days or administering three capsule/tablet each containing 150 to 250 mg of α,β arteether per day for five days.

The kit used in the present invention may be in the form of one or more strip in which the anti-malarial agents are packed individually or in combination. The kit can further comprise an enclosure in the form of a small carton. The instruction is in the form of printed instructions provided inside the carton. The instructions can also be printed on the carton and/or on the strip or strips themselves. The instructions may be in English and/or in any national or regional language.

In the total course of treatment, α,β arteether is administered concurrently with sulfadoxin and pyrimethamine for one day only followed by treatment with α,β arteether for two or four more days.

Thus the novel combination therapy of the present invention comprising the use of α,β-arteether in combination with sulfadoxin and pyrimethamine has distinct advantages in terms of improved effectiveness in the case of multi-drug resistant P. falciparum malaria because of the synergistic antimalarial activity of both the drugs.

The use of the combination kit of the present invention, containing α,β-arteether, sulfadoxan and pyrimethamine is useful from the point of effectiveness in the case of multi-drug resistant P. falciparum malaria/cerebral malaria.

Sulfadoxin and pyrimethamine capsules/tablets and α,β arteether injections or capsules/tablets are obtained commercially or prepared by conventional methods. For instance, injection containing α,β-arteether can be prepared by first dissolving α,β-arteether in neutralized and sterilized arachis oil (75 g/L), filling the solution in injection vial and sealing the injection.

The dosage of the drugs depends on the need of an individual and the dosages described herein are adult doses. However, this invention is not limited to the dosage of the combination regimen described herein and may be varied according to medical advice. Accordingly the specific dosage described in the typical embodiment is only illustrative and non-limiting. Combination kits for other dosage forms are also included in the scope of this invention. The following examples broadly illustrate the nature of this invention and the manner in which it is to be performed without limiting the nature and scope of the invention.

### EXAMPLE 1

### Preparation of Injections of α/β-Arteether

In neutralized refined ground nut oil (one litre) α,β-arteether (75 g) was added and the mixture was stirred for complete mixing). 0.01% BHA as preservative was added to it and the resultant solution was filtered through a membrane filter of 0.45 μm size and filled in a suitable sterile ampoule and sealed. (U.S. Pat. No. 6,326,023 dated Dec. 4, 2001)

### EXAMPLE 2

### Preparation of Capsules/Tablets of α/β-Arteether

Granules of α/βartheether 1.50 g with dicalaum phosphate 3.60 g, lactose 1.50 g, aerosil 0.60 g were made by the conventional methods and lubricated by magnesium stearate 0.15 g and talc 0.15 g. This material was filled in 10 capsules Of alternatively 10 tablets were made of the mixture. It was then packed to avoid contact with moisture before use.

### EXAMPLE 3

### Preparation of Soft Gelatin Capsules of α/β-Arteether

In neutralized refined ground nut oil (one litre) α,β-arteether (75 g) was added and the mixture was stirred for complete mixing. BHA (0.01%) as preservative was added to it and the resultant solution was filtered through a membrane filter of 0.45 μm size and filled in a suitable soft gelatin capsule (0.7 mL in each capsule) with long nozzle and sealed (U.S. Pat. No. 6,326,023)

### EXAMPLE 4

### Chemotherapeutic response of Intramuscularly Administered Arteether (α/β) and Orally Administered Sulfadoxin and Pyrimethamine (Fansidar) in Combination against Multi Drug Resistant P.yoelii nigeriensis Infection in Swiss Mice (20±2 gm)

Experimental: Swiss mice (20±2 g) were inoculated intra peritoneal with 1×10⁶ Plasmodium yoelii nigeriensis (malaria parasite) infected mouse red blood cells. Fansidar was suspended in tween 80 and then in triple distilled water and two different doses, i.e. 1.25-2.5 mg/kg were administered in previously infected Swiss mice. Fansidar was administered by oral route for only 1 day. α/β Arteether was dissolved in neutralized and sterilized ground nut oil and doses i.e. 1.25, 1.78 and 2.5mg/kg were administered by intramuscular route for 3 days. In other groups of infected mice these drugs were given individually for comparison. 2.5, 1.78 and 1.25 mg/kg and α/β arteether were given in combinations with 2.5 and 1.25 mg/kg Fansidar. One group of mice was kept as infected control. After treatment parasitaemia was recorded from tail blood smears of individual mouse. (Table-1)

Results: Results of present experiment suggest that intramuscular arteether (three days treatment) at ⅓^{rd} and ¼^{th} of curative dose (7.5 mg/kg×3) with 1/24^{th} of curative dose of Fansidar (60 mg/kg×1) produced 100% curative efficacy. (Table-1)

Inference: 100% curative effect is achieved with much lower doses when combination of the two drugs are used.

TABLE 1
Synergistic antimalarial effect of α/β arteether (intramuscular) and fansidar
(oral) against multi drug resistant P. yoelii nigeriensis in Swiss mice.

| | Drugs/dose mg/kg | | | | | | | | |
|---|---|---|---|---|---|---|---|---|---|
| Sl. | arteether + fansidar | | Day | Day | Day | Day | Day | Day | |
| No. | ×3 | ×1 day | 4 | 7 | 14 | 18 | 21 | 28 | Cure rate (%) |
| 1 | 2.5 | +2.5 | 6/6 | 6/6 | 6/6 | 6/6 | 6/6 | 6/6 | 100 |
| 2 | 1.78 | +2.5 | 6/6 | 6/6 | 6/6 | 6/6 | 6/6 | 6/6 | 100 |
| 3 | 1.25 | +2.5 | 6/6 | 6/6 | 4/4 | 4/4 | 4/4 | 4/4 | 66.6 |
| 4 | 2.5 | +1.25 | 6/6 | 6/6 | 4/4 | 4/4 | 4/4 | 4/4 | 66.6 |
| 5 | 1.78 | +1.25 | 6/6 | 6/6 | 4/4 | 4/4 | 4/4 | 4/4 | 66.6 |
| 6 | 1.25 | +1.25 | 6/6 | 6/6 | 4/4 | 4/4 | 4/4 | 4/4 | 66.6 |
| 7 | 2.5 | +— | 6/6 | 6/6 | 4/4 | 4/4 | 4/4 | 4/4 | 66.6 |
| 8 | 1.78 | +— | 6/6 | 5/6 | 2/2 | 1/2 | 1/2 | 1/1 | 16.6 |
| 9 | 1.25 | +— | 3/6 | 0/6 | — | — | — | — | 0.0 |
| 10 | — | +2.5 | 6/6 | 3/6 | — | — | — | — | 33.3 |
| 11 | — | +1.25 | 5/6 | 2/6 | 2/2 | 2/2 | 2/2 | 2/2 | 33.3 |

| | | | | |
|---|---|---|---|---|
| 12 | Control | 0/6 | — | 0.0 |

### EXAMPLE 5

### Synergistic Antimalacial effect of Shorter Regimen of α/β Arteether (Intra-Muscular) and Fansidar (Oral) Against Multi Drug Resistant P. yoelii nigerensis in Swiss Mice.

Experimental: Swiss mice (20±2 g) were inoculated intra peritoneal with 1×10⁶ Plasmodium yoelii nigeriensis (malaria parasite) infected mouse red blood cells. Faosidar was suspended in tween 80 and then in triple distilled water and 2.5 mg/kg dose was administered in previously infected Swiss mice. Fansidar was administered by oral route for only 1 day i.e. on day 0 or day 1. α/β Arteether was dissolved in neutralized and sterilized ground nut oil and doses i.e. 7.5 mg/kg×1, ×2, 5 mg/kg×1, ×2 by intramuscular route were given in combination with 2.5 mg/kg ×1 dose of Fansidar. These doses of both the drugs were given individually for comparison. One group of mice was kept as infected control. After treatment parasitaemia was recorded from tail blood smears of individual mouse.

Results: Results of present experiment suggest that intramuscular arteether at the dose of 7.5 mg/kg (1 or 2 days treatment) and 5 mg/kg ×2 day's treatment in combination with Fansidar treatment at 2.5 mg/kg ×1 produced 100% curative efficacy whereas 5 mg/kg×1 with Fansidar 2.5 mg/kg provided 83.3% protection and cure. (Table-.2)

Inference: Thus we get the same 100% curative effect with less number of curative doses of arteether in combination of Fansidar single dose treatment.

TABLE 2
Synergistic antimalarial effect of variable regimen of α/β arteether (intramuscular)
and fansidar (oral) against multi drug resistant P. yoelii nigeriensis in Swiss mice.

| | Drugs/dose mg/kg | | | | | | | | |
|---|---|---|---|---|---|---|---|---|---|
| | α/β | | | | | | | | |
| Sl. | arteether + Fansidar | | Day | Day | Day | Day | Day | Day | |
| No | (day) | (day) | 4 | 7 | 10 | 14 | 21 | 28 | Cure rate (%) |
| 1. | 7.5 × 1 (0) | +2.5 × 1 (0) | 6/6 | 6/6 | 6/6 | 6/6 | 6/6 | 6/6 | 100 |
| 2. | 7.5 × 1 (0) | +2.5 × 1 (1) | 6/6 | 6/6 | 6/6 | 6/6 | 6/6 | 6/6 | 100 |
| 3. | 7.5 × 2 (0, 1) | +2.5 × 1 (0) | 6/6 | 6/6 | 6/6 | 6/6 | 6/6 | 6/6 | 100 |
| 4. | 5.0 × 1 (0) | +2.5 × 1 (0) | 6/6 | 6/6 | 6/6 | 6/6 | 6/6 | 6/6 | 100 |
| 5. | 5.0 × 1 (0) | +2.5 × 1 (1) | 6/6 | 6/6 | 6/6 | 6/6 | 5/5 | 5/5 | 83.3 |
| 6. | 5.0 × 2 (0, 1) | +2.5 × 1 (0) | 6/6 | 6/6 | 6/6 | 6/6 | 6/6 | 6/6 | 100 |
| 7. | 7.5 × 1 (0) | +— | 6/6 | 6/6 | 6/6 | 6/6 | 5/5 | 5/5 | 83.3 |
| 8. | 7.5 × 2 (0, 1) | +— | 6/6 | 6/6 | 6/6 | 6/6 | 6/6 | 6/6 | 100 |
| 9. | 5.0 × 1 (0) | +— | 6/6 | 2/6 | 0/4 | D | — | — | 0.0 |
| 10. | 5.0 × 2 (0, 1) | +— | 6/6 | 6/6 | 6/6 | 6/6 | 5/5 | 5/5 | 83.3 |
| | — | +2.5 × 1 (0) | 6/6 | 6/6 | | 3/6 | 3/3 | 3/3 | 50.0 |
| 11. | — | +2.5 × 1 (1) | 6/6 | 6/6 | 6/6 | 3/6 | 3/3 | 3/3 | 50.0 |

| | | | | |
|---|---|---|---|---|
| 12. | Control | 0/6 | — | 0.0 |

### EXAMPLE 6

### Synergistic Antimalarial effect of Variable Regimen of α/β Arteether (Oral) and Fansidar (Oral) against Multi Drug Resistant P. yoefii nigenesisi in Swiss Mice.

Experimental: Swiss mice (20±2 g) were inoculated intra peritoneal with 1×10⁶ Plasmodium yoelii nigeriensis (malaria parasite) infected mouse red blood cells. Fansidar was suspended in tween 80 and then in triple distilled water and different doses i.e., 5, 2.5 and 1.25 mg/kg were admimstered in previously infected Swiss mice. Fansidar was administered by oral route for only 1 day i.e. on day 0. α/β Arteether was dissolved in neutralized and sterilized ground nut oil and doses i.e. 25 mg/kg×1 ×4, 12.5mg/kg×4 were given orally in combination with 5.0, 2.5 and 1.25 mg/kg ×1 dose of Fansidar. These doses of both the drugs were given individually for comparison. One group of mice was kept as infected control. After treatment parasitaemia was recorded from tail blood smears of individual mouse and their mean parasitaemia was calculated.

Results: Results of present experiment suggest that oral arteether dissolve in oil at the dose of 25 mg/kg×4 and 5, 2.5 and 1.25mg/kg ×1 day's treatment in combination with Fansidar treatment produced 100% curative efficacy whereas 12.5 mg/kg×4 with 5 and 2.5 mg/kg Fansidar also provided 100% curative efficacy. Same dose of arteether i.e. 12.5 mg/kg×4 with 1.25 mg/kg×1 Fansidar could provide 85.7% protection and cure. (Table-3)

Inference: Thus we get the same 100% curative effect with half of curative dose of arteether in combination of 1/48^{th} of Fansidar curative dose. Arteether in ¼^{th} of curative dose is also curative with 1/24^{th} of Fansidar dose. This study clearly indicates the synrgism between the proposed two drugs.

TABLE 3
Synergistic antimalarial effect of variable regimen of α/β arteether
(oral) and fansidar (oral) against multi drug resistant P. yoelii nigeriensis
in Swiss mice.

| Arteether + Fansidar | | Mice remained negative on day | | | | | |
|---|---|---|---|---|---|---|---|
| ×4 | ×1 | 4 | 7 | 14 | 21 | 28 | % Cure rate |
| 25.0 | +1.25 | 7/7 | 7/7 | 7/7 | 7/7 | 7/7 | 100.0 |
| 25.0 | +2.5 | 7/7 | 7/7 | 7/7 | 7/7 | 7/7 | 100.0 |
| 25.0 | +5.0 | 7/7 | 7/7 | 7/7 | 7/7 | 7/7 | 100.0 |
| 12.5 | +1.25 | 7/7 | 7/7 | 6/6 | 6/6 | 6/6 | 85.7 |
| 12.5 | +2.50 | 7/7 | 7/7 | 7/7 | 7/7 | 7/7 | 100.0 |
| 12.5 | +5.0 | 7/7 | 7/7 | 7/7 | 7/7 | 7/7 | 100.0 |
| 25.0 | +— | 7/7 | 7/7 | 4/4 | 4/4 | 4/4 | 57.1 |
| 12.5 | +— | 5/7 | 3/7 | 2/2 | 2/2 | 2/2 | 28.5 |
| — | +1.25 | 7/7 | 2/7 | D | | | 0.0 |
| — | +2.50 | 7/7 | 7/7 | 2/3 | 2/2 | 2/2 | 28.5 |
| — | +5.0 | 7/7 | 7/7 | 6/7 | 6/6 | 6/6 | 85.7 |

### EXAMPLE 7

### Synergistic Sntimalarial effect of α/β Arteether (Oral,Aqueous) and Fansidar (Oral) against Multi Drug Resistant P. yoelii nigenensis in Swiss Mice.

Experimental: Swiss mice (20±2 g) were inoculated intra peritoneal with 1×10⁶ Plasmodium yoelii nigeriensis (malaria parasite) infected mouse red blood cells. Fansidar was suspended in tween 80 and then in triple distilled water and different doses i.e. 2.5 and 1.25 mg/kg were administered in previously infected Swiss mice. Fansidar was administered by oral route for only 1 day i.e. on day 0 α/β Arteether was dissolved in tween 80 and triple distilled water and doses i.e. 24 and 12 and 6 mg/kg×4 were given orally in combination with 2.5 and 1.25 mg/kg ×1 dose of Fansidar. These doses of both the drugs were also given individually for comparison. One group of mice was kept as infected control. After treatment parasitaemia was recorded from tail blood smears of individual mouse and their mean parasitaemia was calculated.

Results: Results of present experiment suggest that oral formulation of arteether dissolved in tween80 and triple distilled water, at the dose of 24 mg/kg ×4 in combination with 1.25 mg/kg ×1 SP treatment produced 83.3 % curative efficacy whereas 12mg/kg×4 arteether with 2.5 and 1.25 mg/kg SP could cause 28.57% curative efficacy.

Inference: Thus we get the 83.3 % curative effect with ⅓rd of curative dose of arteether in combination with 1/48^{th} of SP active dose. Curative dose with oral formulation of arteether is calculated as 72 mg/kg×4. These results again confirm the synergism between the proposed two drugs and antimalarial activity is also proven by oral administration in aqueous preparation of both drugs.

TABLE 4
Synergistic antimalarial effect of α/β arteether
(oral, aqueous) and fansidar (oral) against multi drug
resistant P. yoelii nigeriensis in Swiss mice.

| Arteether + Fansidar | | Mice remained negative on day | | | | | |
|---|---|---|---|---|---|---|---|
| ×4 | ×1 | 4 | 7 | 14 | 21 | 28 | % Cure rate |
| 24 | +1.25 | 14/14 | 14/14 | 12/12 | 12/12 | 12/12 | 83.3 |
| 12 | +2.50 | 7/7 | 7/7 | 2/2 | 2/2 | 2/2 | 28.57 |
| 12 | +1.25 | 7/7 | 7/7 | 2/2 | 2/2 | 2/2 | 28.57 |
| 6 | +2.50 | 7/7 | 6/7 | 1/3 | 1/2 | 1/1 | 14.28 |
| 24 | +— | 7/7 | 3/6 | 1/3 | 1/2 | 1/1 | 14.28 |
| 12 | +— | 3/7 | 0/5 | D | | | 0.0 |
| 6 | +— | 5/7 | 0/3 | D | | | 0.0 |
| — | +1.25 | 6/7 | 1/6 | D | | | 0.0 |
| — | +2.50 | 7/7 | 6/7 | 1/3 | 1/1 | 1/1 | 14.28 |

We claim:

1. A combination kit for the treatment of malaria caused by P. falciparum for a period of two to five days schedule, the kit comprising:
a. individual dose of anti-malarial agents sulfadoxine and pyrimethamine; and
b. individual dose of anti-malarial agent α,β arteether;
c. instruction manual for the administration of the three antimalarial drugs.

2. A kit as claimed in claim 1 wherein the sulfadoxine and pyrimethamine are used in a form selected from the group consisting of capsules, suppositories and tablets.

3. A kit as claimed in claim 1 wherein the α,β arteether is taken in ground nut oil filled in a injection vial or soft gelatin capsule or in the form of capsules or tablets.

4. A kit as claimed in claim 1 wherein the kit comprises:
a. three capsules/suppositories/tablets containing 500 mg of sulfadoxine and 25 mg of pyrimethamine; and
b. three injection each containing 150 mg of α/β arteether or fifteen capsules/tablets contuinng 150-250 mg of α/β arteether.

5. A kit as claimed in claim 1 wherein the kit is in the form of one or more strips in which the anti-malarial agents are packed.

6. A kit as claimed in claim 1 wherein the kit is in the form of a small carton wherein the instruction is in form of printed instruction provided inside the carton.

7. A kit as claimed in claim 1 wherein the instructions are in the form of prints on the carton and/or on the strip or strips.

8. A method for the treatment of malaria caused by P. falciparum comprising administering to a subject malarial medication contained in a combination kit which in turn comprises:
a. individual dose of anti-malarial agents sulfadoxine and pyrimethamnine and
b. individual dose of anti-malarial agent α,β arteether;
c. instruction manual for the administration of the three antimalarial drugs.

9. A method as claimed in claim 8 wherein the sulfadoxine and pyrimethamine are used in a form selected from the group consisting of capsules, suppositories and tablets.

10. A method as claimed in claim 8 wherein the α,β arteether is taken in ground nut oil filled in a injection vial or soft gelatin capsule or in the form of capsules or tablets.

11. A method as claimed in claim 8 wherein the kit comprises:
a. three capsules/suppositories/tablets containing 500 mg of sulfadoxine and 25 mg of pyrimethamine; and
b. three injection each containing 150 mg of α/β arteether or fifteen capsules/tablets containing 150-250 mg of α/β arteether.

12. A method as claimed in claim 11 wherein the method of administration comprises
a. administering three capsules/suppositories/tablets containing 500 mg of sulfadoxine and and 25 mg of pyrimethamine on day one;
b. administering one i.m. injection at a time, each containing 150 mg of α,β arteether per day for two to three days or alternatively administer three tablets/capsules each containing 150 mg-250 mg of α,β arteether per day for three-five days by oral route.

13. A method as claimed in claim 8 wherein the method comprises administering a first anti-malarial agent sulfadoxin, second antimalarial Pyrimethaiine for single day and third anti-malarial agent α/β arteether in predetermined doses and predetermined dose schedule for two to three/five day therapy.
